(19) United States
(12) Patent Application Publication
(10) Pub. No.: US 2022/0193345 A1
(43) Pub. Date: Jun. 23, 2022

(54) DEVICE FOR APPLYING A FLUID

(71) Applicant: Henke-Sass, Wolf GmbH, Tuttlingen (DE)

(72) Inventors: Frank ALTERMANN, Tuttlingen (DE); Daniel SEEH, Immendingen (DE)

(21) Appl. No.: 17/552,272

(22) Filed: Dec. 15, 2021

(30) Foreign Application Priority Data
Dec. 18, 2020 (DE) 102020134181.4

(51) Int. Cl. A61M 5/30; A61M 5/315; A61M 5/31

(52) U.S. Cl. CPC A61M 5/30; A61M 5/31513; A61M 2005/3114; A61M 2005/3103; A61M 2005/3128

(57) ABSTRACT

A device for administering a fluid, wherein, with the valve opened, fluid that is to be administered can be brought through the feed channel into the cylinder, and wherein a movement of the piston counter to the first direction towards the open dispensing end generates a positive pressure in the cylinder, such that, with the first valve opened, the fluid that is to be administered from the cylinder is dispensed via the open dispensing end, wherein, in order to seal the rear end of the cylinder, a first sealing element is arranged between the piston and the cylinder and a second sealing element is arranged spaced apart from the first sealing element along the first direction. The first sealing element can be a single-acting sealing element, and the second sealing element can be a single-acting or double-acting sealing element.

### PRIORITY

This application claims the benefit of German Patent Application No. 102020134181.4, filed Dec. 18, 2020, which is hereby incorporated herein by reference in its entirety.

### FIELD

The present invention relates to a device for administering a fluid, which device can be designed, for example, as a needle-free self-filling syringe with which a liquid medicament, a liquid pharmaceutical product, a liquid vaccine or the like can be administered to animals (in particular by intramuscular administration).

### BACKGROUND

A device for administering a fluid, such as mentioned above, should be as light as possible, and thus able to be carried with one hand for a long period of time by a user, and at the same time should permit the desired injection (in particular the needle-free intramuscular injection).

### SUMMARY

It is an object herein to provide a device for administering a fluid.

A device for administering a fluid has a first sealing element, which is designed as a single-acting sealing element, and a second sealing element, which is spaced apart from the first sealing element, and is designed as a single-acting or double-acting sealing element. Thus, it is possible to provide a good seal of the rear end of the cylinder both with respect to high, dynamic pressures and also to negative pressure. Thus, the first sealing element provides sealing of the rear end of the cylinder with respect to positive pressure, which occurs when the fluid is being administered. Moreover, the provision of the second sealing element has the effect that the rear end of the cylinder is sealed off with respect to negative pressure in the cylinder, which occurs when new fluid is being sucked in for the next administering procedure.

It can thus be said that a tandem seal is provided which is optimized to seal off the rear end of the cylinder to positive pressure (by means of the first sealing element) and to seal off the rear end of the cylinder to negative pressure (by means of the second sealing element).

Here, a single-acting first sealing element is understood in particular as a sealing element which seals off the rear end of the cylinder to positive pressure in the cylinder and which does not seal off the rear end of the cylinder to negative pressure in the cylinder (and thus positive pressure outside at the rear end of the cylinder). Thus, only the positive pressure in the cylinder is sealed off with respect to the rear end.

A single-acting second sealing element is understood in particular as a sealing element which seals off the rear end of the cylinder to negative pressure in the cylinder (and thus to positive pressure outside the cylinder at the rear end). Here, a double-acting second sealing element (or a dual-acting second sealing element) is in particular a sealing element which seals off the rear end of the cylinder with respect to positive pressure and negative pressure in the cylinder.

The first sealing element can have in particular a U-shaped or V-shaped cross section, wherein the opening of the U-shaped or V-shaped cross section points in the direction of the open dispensing end.

The first sealing element can moreover have a tensioning means (e.g. a spring), which presses the two limbs of the U-shaped or V-shaped cross section radially apart from each other.

The material used for the first sealing element can be plastic, a polymer, a fluoropolymer, a polyurethane (e.g. an injection moulded polyurethane or casted polyurethane), an elastomer or PTFE (polytetrafluoroethylene). These materials can contain fillers (e.g. carbon fibres or glass fibres) and/or additives in order to adjust the desired properties of the sealing element. Thus, the first sealing element can be produced from PTFE or a PTFE compound. A PTFE compound is understood in particular as a mixture of PTFE with at least one further substance (e.g. carbon, carbon fibres, carbon black, glass fibres, organic fillers, metals, metal alloys, etc.). If a spring is provided, the latter is preferably made of metal.

The first sealing element can have a ring-shaped sealing part, and a spring element which subjects the ring-shaped sealing part to radially inwardly directed tensioning.

Thus, the ring-shaped sealing part, in the installed state, can be subjected to radially inwardly directed pretensioning.

The ring-shaped sealing part can be produced from a thermoplastic, and the spring element can be produced from an elastomer. The spring element can be designed in particular as an O-ring.

The second sealing element can be designed in the same way as the first sealing element. It is then arranged at 180° relative to the first sealing element (the opening of the U-shaped or V-shaped cross section points away from the open dispensing end and thus points towards the rear end of the cylinder) in order to ensure the desired sealing of the rear end of the cylinder with respect to negative pressure in the cylinder.

It is moreover possible that the second sealing element is designed as a double-acting sealing element. In this case, it can be designed as an O-ring for example.

The same materials as were used for the first sealing element can be used for the second sealing element. For example, elastomers (e.g. fluoroelastomer), PTFE or PTFE compounds can preferably be used.

The first and the second sealing element can be arranged or provided on the cylinder, such that the piston moves relative to the sealing elements. In such an arrangement, the sealing elements are often designated as rod seals.

Alternatively, it is possible that the first and the second sealing element are secured on the piston, such that the piston moves together with the sealing elements in the cylinder. In such an arrangement, the sealing elements are often designated as piston seals.

It is moreover possible to provide a lubricating ring which is spaced apart from the second sealing element along the first direction. The lubricating ring can, for example, be designed as a felt ring or be formed from a sponge-like material. Moreover, the lubricating ring can have oil or grease applied to it. This ensures good lubrication of the piston during the movement in the cylinder.

The cylinder can be designed in one part or in multiple parts and in particular in two parts. In this case, the cylinder can comprise a rear cylinder part, of which the rear end forms the rear end of the cylinder. The first sealing element and second sealing element can be arranged in the rear cylinder part. Moreover, if a lubricating ring is provided, the latter can likewise be arranged in the rear cylinder part.

The rear cylinder part can be connected to a further part of the cylinder (for example the front cylinder part). The connection can be, for example, a releasable connection. For example, it can be provided in particular as a snap-fit closure or screw connection.

The movement of the piston in the cylinder can be effected manually or can be motorized. A motor can be provided which executes both the movement of the piston away from the open dispensing end and also the movement towards the open dispensing end. Alternatively, the motor can effect only the movement of the piston away from the open dispensing end, in which case a tensioning unit (e.g. one or more springs) of a tensioning device is tensioned. For the administering procedure, the tensioning unit is freed, such that the tensioning energy is converted into a movement of the piston towards the open dispensing end. In this way, a high pressure can be built up, which is desired in particular for needle-free administration of the fluid.

The tensioning device can have a ramp which is rotatable by means of a motor and which has a ramp track extending along a helical line, wherein the ramp track ascends from a first plateau along a region of inclination to a second plateau and descends from the second plateau to the first plateau via a transition flank, wherein the ramp track has a transfer region connecting the second plateau and the transition flank. The tensioning device can moreover comprise a roller which is in contact with the ramp track and which is mounted rotatably in a driver, the latter being connected to the piston rod, and therefore, upon rotation of the ramp along a first rotation direction, the ramp track runs below the rotating roller. For the tensioning procedure, the ramp track can be rotated along the first rotation direction such that the roller runs on the region of inclination as far as the second plateau and the piston is thereby moved to its rear end position, wherein the tensioning unit is tensioned. For the dispensing procedure, starting from a contact of the roller with the second plateau, the tensioning device can rotate the ramp track along the first rotation direction until the roller runs over the transfer region and, on account of the tensioned tensioning unit, accelerates towards the first plateau, as a result of which the piston is moved towards the open dispensing end.

The device for administering a fluid can designed as a self-filling syringe. Here, a self-filling syringe is understood in particular as a syringe in which, during or as a result of the movement of the piston away from the dispensing end of the syringe, the fluid that is to be administered is brought into the cylinder, and in which, by means of the movement of the cylinder towards the dispensing end, the fluid that is to be administered is dispensed. The device for administering a fluid is preferably designed as a self-filling syringe for needle-free administration (in particular intradermal, subcutaneous or intramuscular administration) to animals and/or humans.

Of course, the device for administering a fluid can also be designed such that it is designed as a self-filling syringe with a needle at the dispensing end.

The first valve can be designed as a passive valve or as an active valve. Moreover, the second valve can be designed as a passive valve or an active valve. Here, a passive valve is understood in particular as meaning that the opening and closing of the valve is effected by a positive or negative pressure generated in the cylinder on account of the movement of the piston in the cylinder. Here, an active valve is understood in particular as a valve which can be opened and closed by means of an actuator. Here, an opened first or second valve is understood in particular as meaning that the desired fluidic connection between cylinder and dispensing end or feed channel is present. Here, a closed first or second valve is understood in particular as meaning that there is no fluidic connection present between cylinder and dispensing end or feed channel (or the desired fluidic connection, with the valve opened, is interrupted or blocked). The first valve can be designed as a nonreturn valve. Moreover, the second valve can be designed as a nonreturn valve.

The device for administering a fluid can be designed such that a movement of the piston along the first direction generates a negative pressure in the cylinder, and therefore, with the second valve opened, the fluid that is to be administered is sucked into the cylinder via the feed channel. In particular, the negative pressure generated can open the second valve. Moreover, the positive pressure in the cylinder, generated by the movement of the piston counter to the first direction towards the open dispensing end, can open the first valve, as a result of which the fluid that is to be administered from the cylinder is dispensed via the open dispensing end.

It will be appreciated that the features mentioned above and the features still to be explained below can be used not only in the specified combinations but also in other combinations or on their own, without departing from the scope of the present invention.

The invention is explained in even more detail below on the basis of exemplary embodiments, with reference being made to the appended drawings, which likewise disclose features essential to the invention. These exemplary embodiments are only illustrative and should not be construed as restrictive. For example, a description of an exemplary embodiment with a multiplicity of elements or components should not be construed as meaning that all of these elements or components are necessary for implementation. Rather, other exemplary embodiments can also contain alternative elements and components, fewer elements or components, or additional elements or components. Elements or components of different exemplary embodiments can be combined with one another, unless stated otherwise. Modifications and variations that are described for one of the exemplary embodiments can also be applicable to other exemplary embodiments. In order to avoid repetition, the same elements or corresponding elements in different figures are designated by the same reference signs and are not explained several times over. In the figures:

### BRIEF DESCRIPTION OF THE DRAWINGS

FIG. 1 shows a perspective view of an exemplary embodiment of the administering device according to certain embodiments of the invention.

FIG. 2 shows a front view of the cylinder/piston arrangement of the administering device.

FIG. 3 shows a sectional view of the cylinder/piston arrangement along the section line A-A in FIG. 2.

FIG. 4 shows a sectional view of the cylinder/piston arrangement along the section line B-B in FIG. 3.

FIG. 5 shows a sectional view of the cylinder/piston arrangement along the section line C-C in FIG. 4.

FIG. 6 shows an isometric view of the cylinder/piston arrangement, wherein the device is tensioned and the piston is in its rear end position.

FIG. 7 shows an isometric view of the cylinder/piston arrangement, wherein the piston is in its front end position.

FIG. 8 shows a diagram illustrating the profile of the ramp track, wherein the rotation angle α is plotted along the x axis, and the stroke along the longitudinal axis of the piston rod is plotted on the y axis.

FIG. 9 shows a sectional view of the piston/cylinder arrangement in the tensioned state according to FIG. 6.

FIG. 10 shows a side view of the piston/cylinder arrangement in which the piston is in its front end position.

FIG. 11 shows a sectional view of the piston/cylinder arrangement along the section line D-D in FIG. 10.

FIG. 12 shows an enlarged sectional view of the front part.

FIG. 13 shows an enlarged detailed view of the detail E according to FIG. 12, wherein the piston is positioned nearer the open dispensing end when compared to the view in FIG. 12.

FIG. 14 shows an enlarged view of the ring cross section in an embodiment of the first sealing element.

FIGS. 15-18 show enlarged views of the ring cross section in further embodiments of the first sealing element.

### DETAILED DESCRIPTION

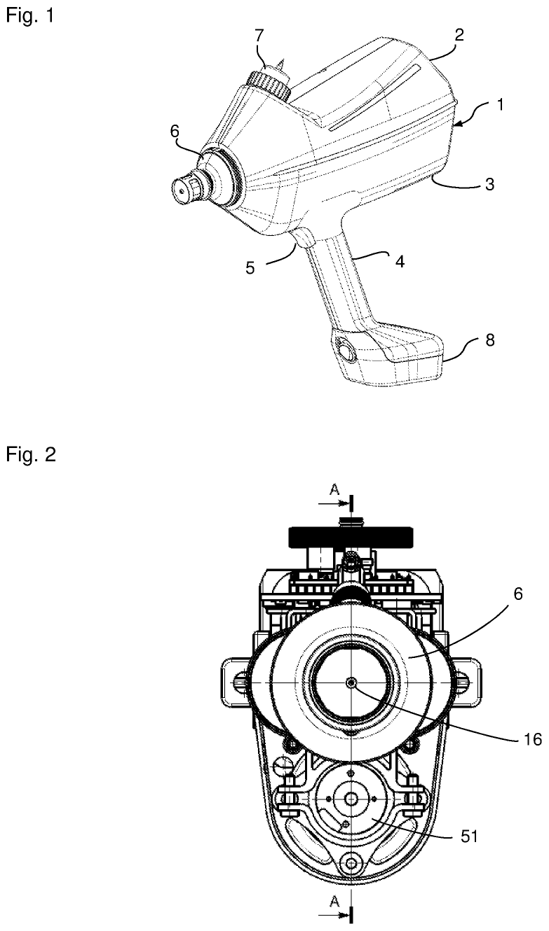

In the exemplary embodiment shown in FIG. 1, the device 1 according to certain embodiments of the invention for administering a fluid (e.g. a liquid) comprises a housing 2 with a main portion 3 and a handle portion 4. The handle portion 4 is designed such that a user can hold the device 1 by grasping the handle portion 4. Furthermore, the handle portion 4 has a trigger 5 for actuating the device 1. A dispensing region 6 is formed at the front end of the main portion 3. Furthermore, at the top region of the main portion 3, the device 1 has an attachment 7 to which, for example, a hose or a container can be connected. The fluid that is to be administered can be delivered via the hose. Similarly, the fluid that is to be administered can be held in the container.

At its end facing away from the main portion 3, the handle portion 4 transitions into a base 8 in which, for example, a power supply (for example an accumulator) for the device 1 can be contained.

In the exemplary embodiment described here, the device 1, which can also be designated as administering device 1, is designed for the needle-free administration of the fluid to an animal. The administration preferably involves the intramuscular injection of the fluid which, for example, can be a pharmaceutical product, a vaccine or the like.

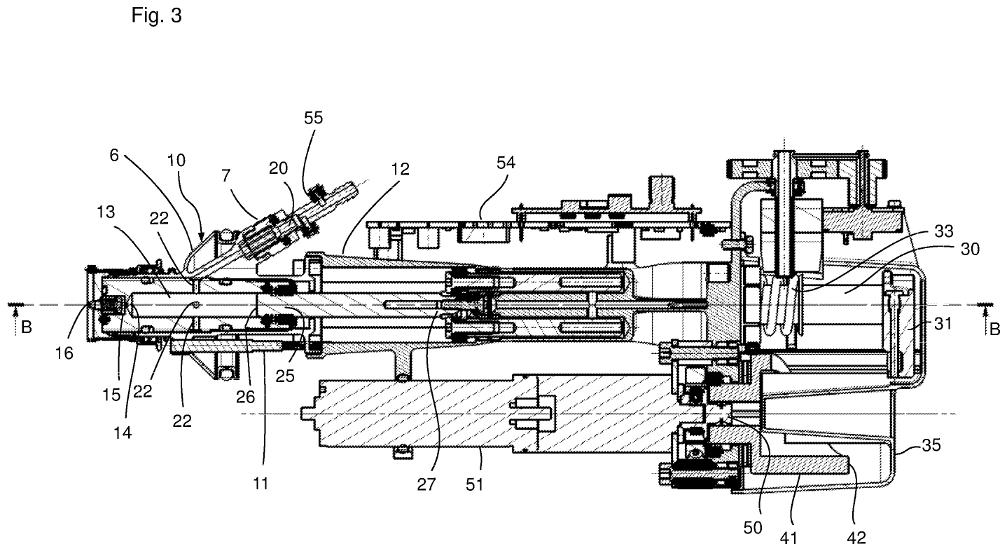

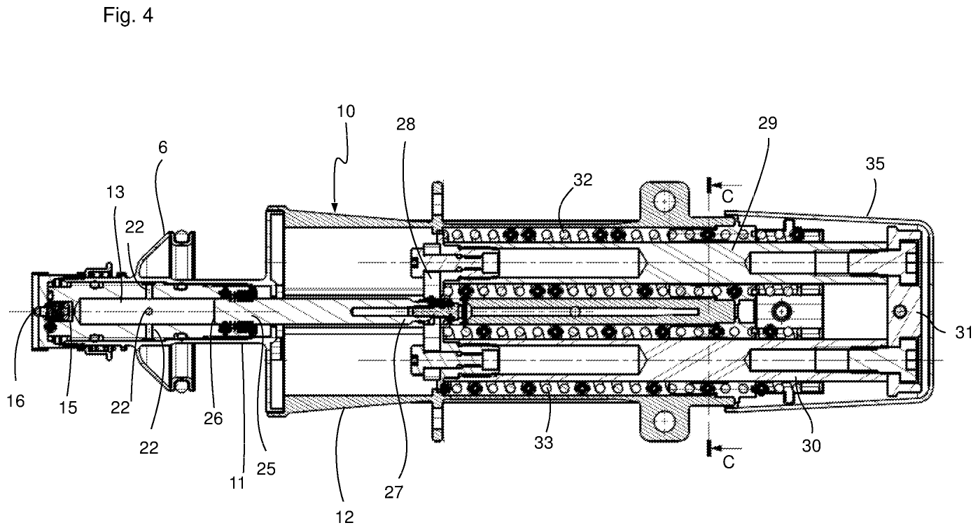

The administering device 1 has a cylinder/piston arrangement 10 (FIGS. 3 and 4) described in more detail below and is of the self-filling type, such that a movement of the piston towards the dispensing region 6 causes a discharge of the fluid, and an opposite movement of the piston causes the cylinder to fill with the fluid for the next discharge procedure.

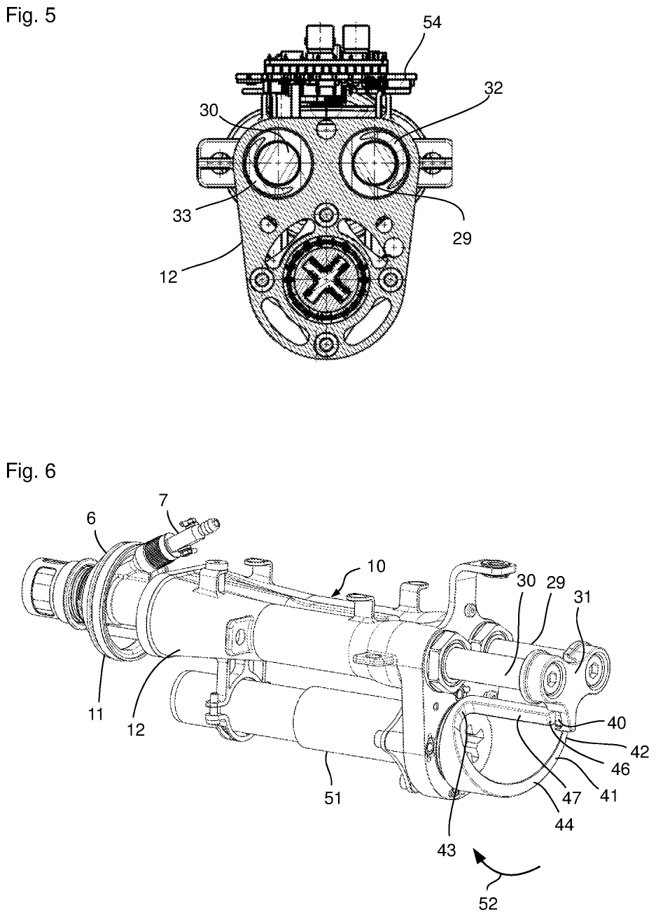

FIGS. 2 to 5 show the whole cylinder/piston arrangement 10 without the housing 2. The cylinder/piston arrangement 10 comprises a front part 11 and, connected to the latter, a rear part 12. The front part 11 comprises a cylinder 13 for receiving the fluid, which cylinder 13 has an open dispensing end 14 in which a first nonreturn valve 15 sits, the latter being fluidically connected to a nozzle 16. The first nonreturn valve 15 can also be seen clearly in the enlarged sectional view of the front part 11 in FIG. 12 and is designed so as to permit dispensing of the fluid from the cylinder 13 via the first nonreturn valve 15 and the nozzle 16. Suctioning of air or liquid via the nozzle 16 and via the first nonreturn valve 15 is not possible. The nonreturn valve 15 closes in this direction.

Also formed at the front part 11 is the attachment 7, in which a second nonreturn valve 20 (FIG. 12) sits, the latter permitting a fluidic connection of the attachment 7 to the cylinder 13 and blocking a fluidic connection in the opposite direction. The attachment 7 has a channel 21 (or feed channel 21) which opens into the cylinder 13 via a plurality of radial bores 22.

The second nonreturn valve 20 can be designated as an inlet valve, and the first nonreturn valve 15 can be designated as an outlet valve.

A piston 25 with a piston end 26 pointing towards the open dispensing end 14 is guided in the cylinder 13, wherein the piston 25 is in its rear end position in the sectional views in FIGS. 3, 4 and 12. In this position, the cylinder 13 is filled with the fluid that is to be administered. The piston 25 is here designed as a rod of constant cross section, wherein the part of the rod that is moved to and fro in the cylinder 13 is designated as the piston 25, and the part of the rod that protrudes rearwards from the cylinder 13 is designated as the piston rod 27.

The rear end (shown clearly in FIG. 4) of the piston rod 27 pointing away from the open dispensing end 14 is connected by a plate 28 to a first guide rod 29 and a second guide rod 30, which extend parallel to each other and parallel to the piston 25 and are guided in the rear part 12. Those ends of the guide rods 29 and 30 facing away from the plate 28 are connected to a driver 31.

Moreover, a compression spring 32, 33 (e.g. a helical spring) is arranged for each guide rod 29 and 30, the front ends of the compression springs 32, 33 each bearing on the plate 28, and their rear ends each bearing on an abutment of the rear part 12. In the position of the piston 26 shown in FIGS. 3 and 4, the springs 32, 33 are tensioned.

Arranged at the rear end of the rear part 12 is a cover 35, which is not shown in the isometric view of the cylinder/piston arrangement 10 according to FIG. 6 in order that the driver 31 can be clearly distinguished. The driver 31 has a rotatably mounted roller 40, wherein the rotation axis of the roller 40 extends substantially perpendicular to the longitudinal axis of the rod-shaped piston 25.

The roller 40 runs on a ramp track 41 of a ramp 42 that rotates under the roller 40, wherein the ramp track 41 has a single winding, as can be seen in particular in FIGS. 6 to 8.

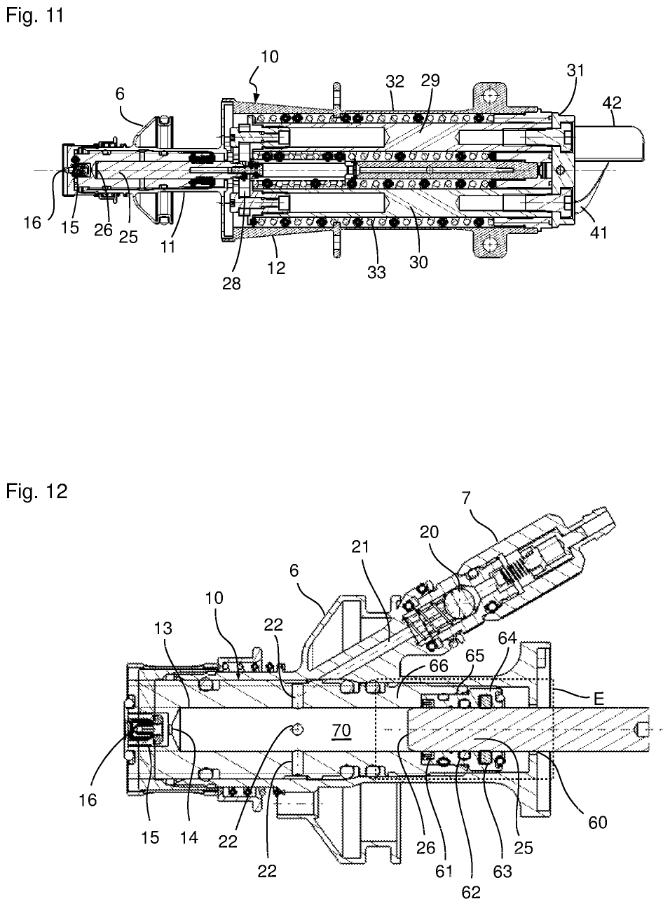

In FIG. 8, the rotation angle α is plotted with respect to the pitch difference z parallel to the longitudinal direction of the piston rod 25, wherein it is assumed that, at a rotation angle of α0=0°, the smallest pitch height z0 is present and the piston 26 is thus in its front end position, in which the distance of the front piston end 26 from the open dispensing end 14 is minimal. This position of the piston 25 is shown for example in the sectional view according to FIG. 11.

The ramp track 41 has a lower plateau 43, which is adjoined by a region of inclination 44, the latter extending as far as the upper plateau 45. The upper plateau 45 is adjoined by a transfer region 46, which merges into a transition flank 47 (rotation angle α1), which in turn leads to the first plateau 43. The rotation angle range from α0 to α2 thus equals 360°.

The transition flank 47 is distinguished by the fact that it runs virtually vertically, since it extends from the height z1 to the height z0 at a rotation angle (here α2). The transfer region 46 is thus the rotation angle range at which the height z1 decreases continuously starting from the upper plateau 45, until the rotation angle α2 (=transition flank 47) is reached. Thus, the rotation angle range of α1 to α2 covers the transfer region 46.

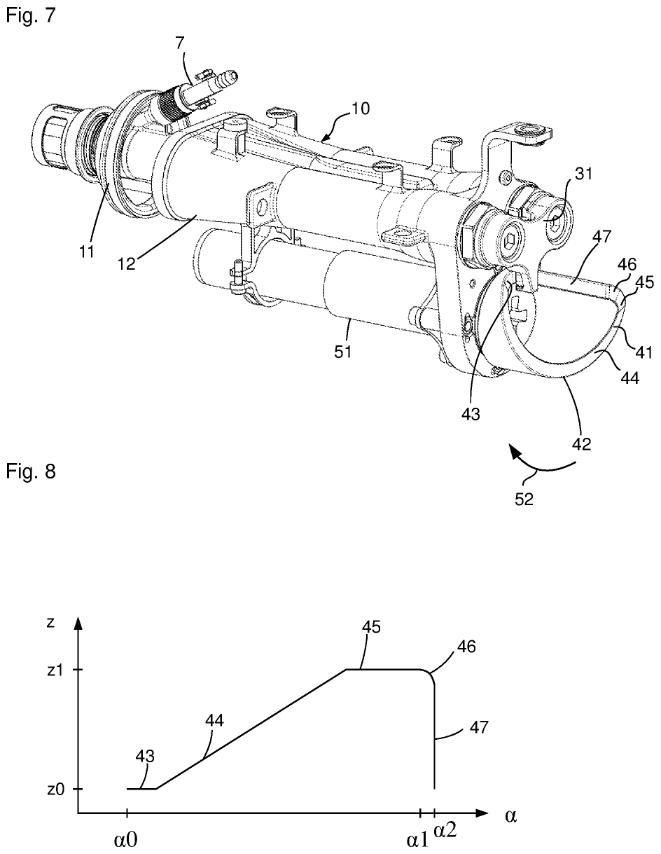

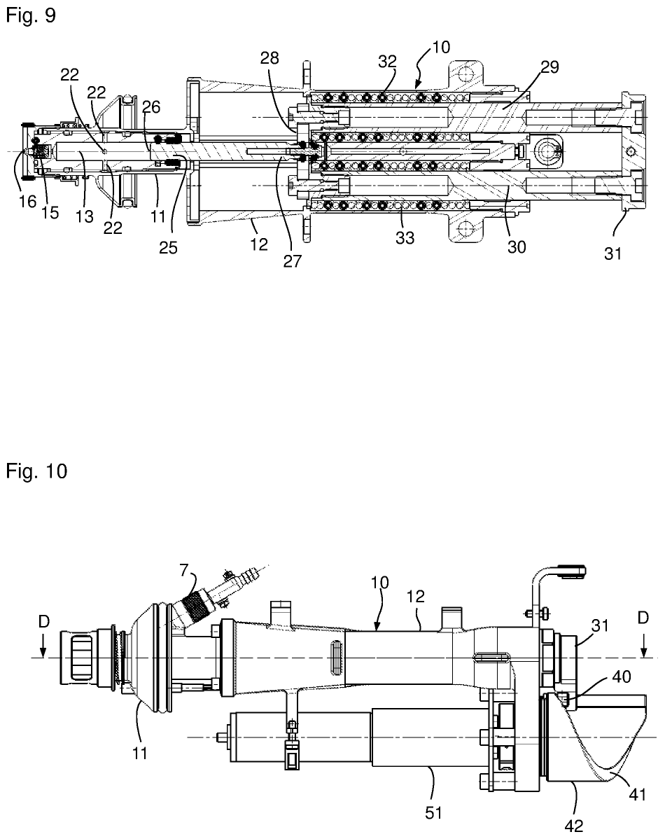

The ramp 42 is connected by a coupling 50 to a motor 51 (FIG. 3) which rotates the ramp 42 in a first rotation direction 52 (FIGS. 6 and 7). If, starting from the position shown in FIG. 6 in which the cylinder/piston arrangement 10 is tensioned, the motor 51 now rotates the ramp 42 further in the first rotation direction 52 (since a user has actuated the trigger 5), the roller 40 runs over the transfer region 46 and then descends along the transition flank 47 in the direction of the lower plateau 43, since the tensioned compression springs 32 and 33 accelerate the plate 28 in the direction of the open dispensing end 14, as a result of which the piston 25 connected to the plate 28 is likewise moved towards the front dispensing end 14, and the fluid contained in the cylinder 13 is thereby discharged, via the first nonreturn valve 15 and the nozzle 16, for intramuscular injection into an animal. The administering device 1 is designed such that the fluid safely penetrates the skin and is administered into the muscle lying beneath the latter. The front piston end 26 is then in its front end position, as is shown for example in the sectional view in FIG. 11. The administering device 1 is preferably configured such that, in the front end position of the front piston end 26, the driver 31 bears on the rear end of the rear part 12, as a result of which the rear end of the rear part 12 forms an abutment for the driver 31. In this position, there is still a desired minimal distance between the roller 40 and the ramp track 41, such that the lower plateau 43 of the ramp track 41 is not reached by the roller 40. It is thus possible to prevent a situation where the roller 40, at the end of the discharging procedure, strikes the ramp track 41, which could cause damage to the roller 40.

After the discharging procedure, the ramp 42 is rotated again in the first rotation direction 52 by means of the motor 51, such that, as soon as the roller 40 makes contact with the ramp track 41 in the region of inclination 44, further rotation has the effect that the driver 31 is moved along the longitudinal direction of the piston 25 away from the open dispensing end 14, as a result of which the compression springs 32, 33 are tensioned again and reach their maximum tensioning when the roller 40 reaches the upper plateau 45. On account of the mechanical connection of the driver 31 to the guide rods 29 and 30, to the plate 28 and to the piston rod 25, this movement of the driver 31 has the effect that the piston 25 and thus the front piston end 26 are also moved in a direction away from the open dispensing end in the cylinder 13, and a negative pressure is thus built up. As soon as the built-up negative pressure is so great that the inlet valve 20 opens, the fluid is sucked through the inlet valve 20 and the radial bores 22 into the cylinder 13, such that the cylinder 13 is filled with the fluid.

When the roller 40 (which can also be designated as a cam or a barrel) has reached the upper plateau 45, the motor 51 stops, such that the cylinder/piston arrangement 10 is tensioned and therefore the administering device 1 is ready for the next administering procedure, which can be carried out by actuating the trigger 5.

The plate 28, the springs 32, 33 and guide rods 29, 30, the driver 31 with the roller 40, and the ramp 41 form, together with motor 51 and coupling 50, a tensioning device S for tensioning the cylinder/piston arrangement 10.

The administering device 1 moreover comprises a control unit 54 for controlling the motor 51 and all the other electrical components of the device 1. FIG. 3 shows a printed circuit board with the control unit 54.

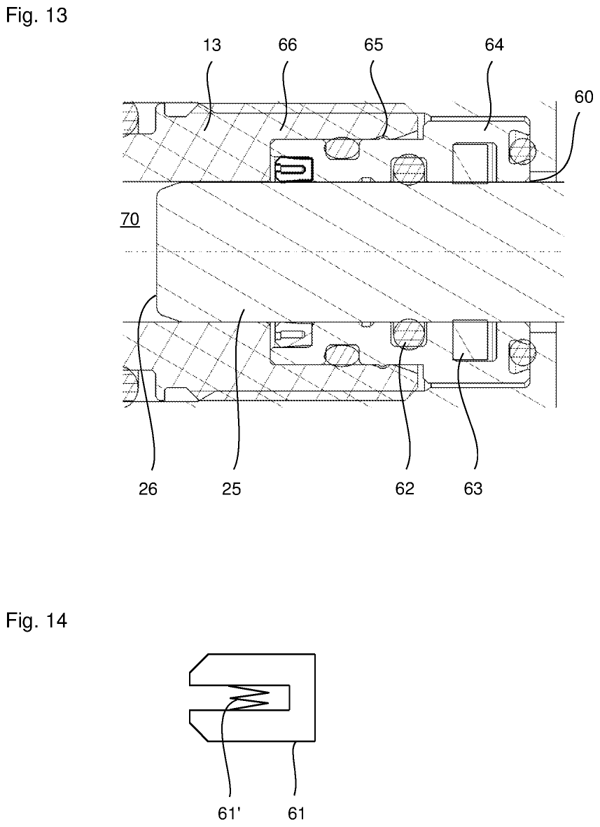

In the case of the administering device 1, it is important that the rear end 60 of the cylinder 13 (FIGS. 12 and 13) is well sealed, because the intended use of the administering device 1 causes high, dynamic pressures and also negative pressure. In the cylinder 13, which has a pressure space 70, there prevails both positive pressure (upon administration of the fluid) and negative pressure (upon suctioning of new fluid for the next administering procedure). The speed of the piston 25 during the suctioning is much slower than it is during the administering. Standard O-ring seals are generally not designed for high, dynamic pressures, since such O-rings quickly become worn. In addition, abrasion of the O-ring seals can have the disadvantage of clogging the outlet valve 16, for example. This can lead to malfunctioning and, consequently, to the repair work required for the latter, and also to an undesirably higher outlay on maintenance.

A first sealing element 61 and a second sealing element 62 can be spaced apart from the latter along the first direction (from left to right in FIG. 12). The first sealing element 61 is designed as a single-action sealing element or a single-acting sealing element, which seals off the rear end 60 of the cylinder 13 with respect to a positive pressure in the cylinder 13. The first sealing element 61 can be ring-shaped and have a U-shaped cross section (or V-shaped cross section), wherein the open end of the U-shaped cross section (or V-shaped cross section) is open towards the front end 14 or the open dispensing end 14. With a positive pressure in the pressure space 61, the two limbs of the U-shaped cross section (or V-shaped cross section) of the first sealing element 61 are then pressed radially apart from each other, such that the desired sealing action can be ensured. The first sealing element 61 can have a spring element 61′ in the U-shaped cross section (or V-shaped cross section), which spring element 61′ already presses the two limbs of the U-shaped cross section (or V-shaped cross section) radially apart from each other, in order to strengthen the desired sealing action, as is shown schematically in FIG. 14.

However, since the single-acting sealing element 61 does not seal off, or only poorly seals off, the rear end 60 of the cylinder 13 with respect to negative pressure in the pressure chamber 70, the second sealing element 62 is provided. In the exemplary embodiment described here, the second sealing element 62 is designed as a double-action sealing element 62 or a double-acting sealing element 62 (it can be an O-ring seal for example), which seals off the rear end 60 of the cylinder 13 with respect to negative pressure in the pressure space 70.

Since the first sealing element 61 seals off the rear end 60 when there is a positive pressure in the pressure space 70, then, compared to previously known solutions, the pressure on the second sealing element 62 in the event of a positive pressure in the pressure chamber 70 is no longer so high, and therefore the undesired wear on such an O-ring seal 62 is greatly reduced.

Moreover, a lubricating ring 63 can also be provided which is arranged between the cylinder 13 and the piston 25. It can be, for example, a felt ring or a sponge-like material. The lubricating ring 63 can have oil or grease applied to it for example, in order to ensure the best possible lubrication of the piston 25.

In the embodiment shown in FIGS. 12 and 13, the first sealing ring 61 and second sealing ring 62 and the optional lubricating ring 63 are arranged in a fixed position on the cylinder 13, such that the piston 25 moves relative to the first sealing ring 61 and second sealing ring 62 and to the optional lubricating ring 63. It is of course also possible to arrange the first sealing ring 61 and/or the second sealing ring 62 (preferably both sealing rings 61 and 62) in a fixed position on the piston 25, such that the piston 25 moves together with the sealing rings 61 and 62. The lubricating ring 63 can also be arranged in a fixed position on the piston 25.

As can be clearly seen from the enlarged detailed view in FIG. 13, the first sealing ring 61 and second sealing ring 62 and the optional lubricating ring 63 are arranged in a rear cylinder part 64, of which the rear end also forms the rear end 60 of the cylinder 13, and which is connected to the front cylinder part 66 of the cylinder 13 by a snap-fit closure 65.

This results in a compact cylinder 13 which can be removed in its entirety from the device. This compact cylinder 13 can then be divided into its parts and, for example, only the rear cylinder part 64 along with the sealing rings 61 and 62 and the lubricating ring 63 need be replaced. It is of course also possible to replace only the first sealing ring 61 and second sealing ring 62 and the optional lubricating ring 63 during maintenance and to use the rear cylinder part 64 again.

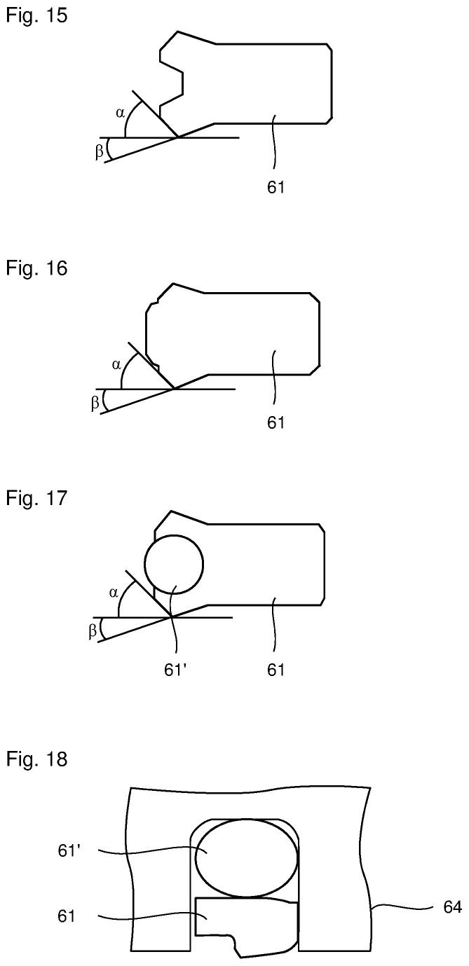

In the embodiment shown in FIG. 15, the first sealing ring 61 is designed as a grooved ring. In this case in particular, the obtuse-angled sealing edge can have a greater angle α with the grooved-ring surface than the outer face of the sealing edge with the shallower angle β. Hard polyurethane can be used as material.

FIG. 16 shows a modification of the grooved ring of FIG. 15.

In FIG. 17, the grooved ring of FIG. 15 is modified such that it is in two parts and additionally has the elastic O-ring 61′, which presses the sealing edges radially outwards and ensures the desired pretensioning.

FIG. 18 shows a further possible embodiment of the first sealing element 61. In this embodiment, a sealing ring 61 (e.g. of PTFE or PTFE compound) is provided which has an elastomer O-ring 61′ on its outer face. The elastomer O-ring 61′ serves to press the sealing ring 61 onto the piston 25. For this purpose, the first sealing element 61 so designed sits (with the elastomer O-ring 61′) in a corresponding receiving groove in the rear cylinder part 64.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products. Moreover, features or aspects of various example embodiments may be mixed and matched (even if such combination is not explicitly described herein) without departing from the scope of the invention.

What is claimed is:

1. A device for administering a fluid, comprising:
a cylinder, which has an open dispensing end;
a piston, which is displaceable in the cylinder between a front end position and a rear end position, and is connected to a piston rod which, along a first direction, protrudes from a rear end of the cylinder opposite the open dispensing end;
a first valve closing the open dispensing end;
a feed channel connected to the cylinder; and
a second valve closing the feed channel,
wherein, with the second valve opened, fluid that is to be administered can be brought through the feed channel into the cylinder,
wherein a movement of the piston counter to the first direction towards the open dispensing end generates a positive pressure in the cylinder, such that, with the first valve opened, the fluid that is to be administered from the cylinder is dispensed via the open dispensing end,
wherein, in order to seal the rear end of the cylinder, a first sealing element is arranged between the piston and the cylinder and a second sealing element is arranged spaced apart from the first sealing element along the first direction,
wherein the first sealing element is configured as a single-acting sealing element, which seals off the rear end of the cylinder with respect to positive pressure in the cylinder, and
wherein the second sealing element is configured as a single-acting or double-acting sealing element, which seals off the rear end of the cylinder at least with respect to negative pressure in the cylinder.

2. The device according to claim 1, wherein the first sealing element is ring-shaped and has a U-shaped or V-shaped ring cross section, and wherein an open end of the U-shaped or V-shaped ring cross section points towards the open dispensing end.

3. The device according to claim 2, wherein the first sealing element comprises a spring element, which is arranged in the U-shaped or V-shaped ring cross section and which presses two limbs of the U-shaped or V-shaped ring cross section apart from each other.

4. The device according to claim 1, wherein the first sealing element has a ring-shaped sealing part and a spring element, which subjects the ring-shaped sealing part to radially inwardly directed tensioning.

5. The device according to claim 4, wherein the ring-shaped sealing part is formed of a thermoplastic, PTFE or a PTFE compound, and the spring element is formed of an elastomer.

6. The device according to claim 1, wherein the second sealing element is configured as an O-ring seal.

7. The device according to claim 1, wherein the first sealing element and the second sealing element are arranged in a fixed position on the cylinder, such that the piston moves relative to the first sealing element and second sealing element.

8. The device according to claim 1, wherein the cylinder has a front cylinder portion and, connected releasably to the latter, a rear cylinder portion, the rear end of which forms the rear end of the cylinder, wherein the first sealing element and second sealing element are arranged in a fixed position on the rear cylinder portion.

9. The device according to claim 8, wherein the rear cylinder portion is connected releasably to the front cylinder portion.

10. The device according to claim 9, wherein the releasable connection of the rear cylinder portion to the front cylinder portion is a snap-fit closure.

11. The device according to claim 1, wherein a lubricating ring is arranged spaced apart from the second sealing element in the first direction.

12. The device according to claim 1, wherein the first valve is configured as a nonreturn valve and/or the second valve is configured as a nonreturn valve.
